(19) United States
(12) Patent Application Publication
(10) Pub. No.: US 2016/0015846 A1
(43) Pub. Date: Jan. 21, 2016

(54) METHOD FOR IMPROVING CONDITIONS IN A POULTRY GROW OUT FACILITY PRIOR TO PLACING CHICKS BY MEANS OF OZONE

(71) Applicant: Rafael Correa, Salisbury, MD (US)

(72) Inventor: Rafael Correa, Salisbury, MD (US)

(21) Appl. No.: 14/720,337

(22) Filed: May 22, 2015

Related U.S. Application Data
(60) Provisional application No. 62/001,864, filed on May 22, 2014.

(51) Int. Cl. A61L 2/20; A01M 13/00; A01K 31/22

(52) U.S. Cl. CPC A61L 2/202; A01K 31/22; A01M 13/00

(57) ABSTRACT

A method and system for the decontamination of poultry houses between flocks where the building is evacuated of all animals and people, and the poultry house, in particular the bedding, is saturated with high levels of ozone, preferably using a portable and transportable ozone supply and delivery system. A tarp may be used over top of the bedding and the ozone delivered beneath the tarp to concentrate the ozone to the bedding. The system may also be used to decontaminate water lines in the poultry house.

### BACKGROUND OF THE INVENTION

1. Field of the Invention

This invention relates to the poultry industry, and more particularly to methods and systems for the decontamination of poultry houses.

2. Description of the Background

The poultry industry has always faced the challenge of placing day old chicks coming from a sterile environment into a poultry house that contains high levels of residual contamination in the bedding and walls from previous flocks.

Today the poultry industry has two choices to improve the conditions of the poultry house and therefore the health of the birds: increase the chick's protection or reduce the risks associated with the challenge.

In order to increase chick protection, the industry typically protects the chicks by the use of injected and/or sprayed biologicals (vaccines) and chemical treatment of the bedding to control contaminants including darkling beetles and their larva population, which are major disease carriers and are consumed by the chicks, causing cross contamination of the flock.

In order to reduce the risks associated with challenge, today's poultry house operations typically allow the house a die-down period of fourteen days between flocks. This time permits the bedding material and feces to dry and solidify (cake). This drying process reduces the surface bacterial load, improving the conditions for receiving the new born chicks. The bedding material typically used, however, is highly absorbent making it very difficult to dry throughout its depth. As long as there is available water, conditions for bacteria production exist.

The efficacy of this drying process is also dependent on weather, season, and geographical location.

The recommended fourteen day period incurs an adverse effect in operating conditions. Over this period of time, the water lines are without circulation and are internally building a bio-film. This bio-film is neither easy to remove nor will it flush out by simply moving higher volumes of water through the lines.

Additionally, the market demands do not always allow for this fourteen day production break, and growers are often forced to place new born chicks in conditions that are far from optimal. As a result there are several potential consequences such as high vaccine cost, early mortality, low feed conversion due to diseases such as, coccidiosis, clostridium, Laryngotracheitis, runting-stunting syndrome, etc.

The use of ozone has been proposed as a way to decontaminate poultry houses, see: U.S. Pat. No. 6,325,971 B1 by inventor Charles Hayes and U.S. Published Patent Application NO. 2004/0141874 A1 by inventor Phillip Mullinax. Both documents describe permanent ozone decontamination installations, and Mullinax clearly describes using the system to provide ozonated air and water to growing animals.

OSHA (Occupational Safety and Health Administration) regulates the use and exposure to ozone in the presence of human's, pets/birds, and animals to a maximum of 0.05 PPM for a maximum period of 8 hours per day and a maximum of 5 days a week.

The low level of ozone concentration required by OSHA in the presence of persons and/or living animals would be impossible to control and monitor in a poultry growing facility. Ozone gas is extremely unstable, has a short life, and naturally wants to revert back to oxygen. These effects depends on ambient temperature; the higher the temperature, the shorter the ozone's life. Therefore, considering OSHA regulations on the use of ozone, the Haynes and Mullinax inventions have limited usage.

### SUMMARY OF THE INVENTION

The present invention describes a method to “shock treat” a poultry house and water lines between flocks, utilizing portable ozone producing and distributing equipment.

Poultry houses are preheated to receive the chicks at 96° to 98° Fahrenheit, with minimum ventilation to remove humidity. As the chicks grow, the temperature is reduced and the ventilation increased. Outside temperature, day/night temperature differentials and relative humidity, etc., are dynamic variables affecting a poultry house operation, making it impossible to meet OSHA's ozone exposure regulations standards. Given the above mentioned conditions, i.e., the regulated exposure of the chicks and the varying and fluctuating conditions of each house, a concentration of 0.05 PPM of ozone in the air is both impossible to maintain and has not been demonstrated to be effective in killing the variety and volume of organisms found in a poultry house environment.

The present invention presents a solution to all the above mentioned challenges and shortcomings.

According to an embodiment of the invention, the present invention includes a method to treat a poultry house in between flocks, preferably by means of a mobile vehicle and/or trailer, equipped with high volume ozone production equipment, an oxygen concentrator or bottled oxygen, an electric generator capable of powering the oxygen concentrator and the ozone machine, portable air blowers, air socks and sufficient ozone resistant hose to connect and carry the ozone from the vehicle outside the poultry house to the portable air blowers inside the poultry house.

According to another embodiment of the invention, a poultry house is “shock treated” by using high levels of ozone, i.e., generally greater than 3 ppm, preferably greater than 5 ppm, more preferably greater than 10 ppm, and most preferably greater than 20 ppm. In order to perform this treatment the poultry house must be closed, and all ventilation equipment shut off. There must be no animals, birds or people inside the house during the shock treatment, and the service will preferably be performed by a factory trained certified technician equipped with the proper safety equipment.

According to the invention, the levels used for the ozone shock treatment will have the desired effect on killing the high levels of pathogens and also have an adverse effect on the darkling beetles and their larvae, flies and their larvae, rodents and snakes that typically are attracted to the environment in an active poultry house.

Therefore, there is provided according to an embodiment of the invention, a method for treating a contaminated poultry house comprising delivering greater than 3 ppm ozone, preferably greater than 5 ppm, more preferably greater than 10 ppm, and most preferably greater than 20 ppm, to the poultry house using portable ozone equipment requiring no permanent installations. According to various embodiments, the ozone may act as a disinfectant, an insecticide and/or a deodorizer.

According to a preferred embodiment of the invention, the ozone is generated using portable and mobile equipment. According to another embodiment of the invention poultry house is empty of live poultry during treatment.

According to another embodiment, the flow and concentration of the ozone may be controlled, optionally by the ozone-air mixer, according to temperature and humidity conditions in the poultry house to maximize decontamination efficiency.

According to another embodiment, the ozone is distributed inside a poultry house environment by means of fans and air socks and a continuous mixing of ozone gas. According to a preferred embodiment, a tarp is placed over the poultry house bedding and a mixture of air and ozone is pumped under the tarp to insure penetration of the ozone gas within the bedding material.

According to yet another embodiment of the invention, there is provided a system for the treatment of contaminated poultry houses having a mobile platform, an oxygen supply mounted on the mobile platform; an ozone generator mounted on the mobile platform and configured to receive oxygen from the oxygen supply; an ozone-air mixer mounted on said mobile platform and configured to receive ozone from the ozone generator and to mix air with ozone; a blower configured to attach directly or indirectly (via tubing or conduit) to the ozone-air mixer, and an air sock configured to attach directly or indirectly (via tubing) to said blower; where the blower configured to draw an ozone-air mixture from the ozone-air mixer and force it through the air sock to deliver an ozone-air mixture to a poultry house.

### DETAILED DESCRIPTION OF THE INVENTION

A mobile platform or vehicle, open (e.g., flatbed) or enclosed, is configured to carry an oxygen supply (e.g., oxygen concentrator or bottled oxygen) for ozone production, an ozone generator or other ozone production device connected to oxygen supply, an ozone-air mixer in communication with the ozone production device for mixing air with the ozone produced by the ozone production device, high CFM portable fans or other blower devices for forcing air from the ozone-air mixer into a poultry house, flexible tubing for carrying the ozone-air mixture from the blower devices into the poultry houses and air socks or other portable gas distribution assembly for distributing the ozone-air mixture in the poultry house, and, optionally, a power generator.

According to an embodiment of the invention, the poultry house is evacuated of all poultry, poultry house ventilation systems are turned down or off, the mobile platform or vehicle is towed or driven to a location adjacent to the poultry house, and the air socks are laid on top of or below the bedding material. The air socks are connected to the blower device either directly or via flexible tubing, and the blower devices are connected to the ozone-air mixer directly or indirection through flexible tubing. The ozone generation machine takes oxygen from the oxygen supply, generates ozone and delivers it to the ozone-air mixer. The blowers draw the ozone-air mixture from the mixer and deliver to the air socks lying on or under the bedding to distribute high concentrations of ozonized air evenly throughout the poultry house.

According to a preferred embodiment, a tarp may be placed over the bedding material, wall-to-wall, and the air sock or other gas delivery apparatus inject ozone saturated air under the tarp creating a low pressurized condition under the tarp forcing ozone saturated air to penetrate the permeable contaminated bedding material. Once the treatment is completed, the ozone treatment system can be disassembled, the dismounted portions mounted/stored back on the mobile platform/vehicle, and moved to the next poultry house for treatment.

The house will preferably remain closed for one hour after the shock treatment has been completed to ensure that all the ozone (O₃) has reverted to oxygen (O₂).

During the service and as part thereof, using separate ozone production equipment the water lines will be flushed with high concentration of ozone in the water to remove bio-film and calcium/iron residue residing in the water lines.

According to the invention, the concentration of the ozone delivered to the poultry house may be set (using, e.g., the ozone-air mixer) based on the level of contamination to achieve a desired level of decontamination in the air, water and/or bedding for the incoming day old chicks that have not yet developed their immune system.

1. A method for treating a contaminated poultry house comprising delivering greater than 3 ppm ozone to the poultry house using portable ozone equipment requiring no permanent installations.

2. The method of claim 1 in which the ozone is used as a disinfectant, insecticide and deodorizer.

3. The method of claim 1 in which the ozone is generated using portable and mobile equipment.

4. The method of claim 1 in which the poultry house is empty of live poultry.

5. The method of claim 1 comprising controlling the flow and concentration of ozone according to temperature and humidity conditions in the poultry house to maximize decontamination efficiency.

6. The method of claim 1 wherein the ozone is distributed inside a poultry house environment by means of fans and air socks and a continuous mixing of ozone gas.

7. The method of claim 1 further comprising placing a tarp is placed over the poultry house bedding and a mixture of air and ozone is pumped under the tarp to insure penetration of the ozone gas within the bedding material.

8. A system for the treatment of contaminated poultry houses comprising:
a mobile platform;
an oxygen supply mounted on said mobile platform;
an ozone generator mounted on said mobile platform and configured to receive oxygen from said oxygen supply;
an ozone-air mixer mounted on said mobile platform and configured to receive ozone from said ozone generator and to mix air with said ozone;
a blower configured to attach directly or indirectly to said ozone-air mixer, and
an air sock configured to attach directly or indirection to said blower;
said blower configured to draw an ozone-air mixture from said ozone-air mixer and force it through said air sock to deliver said ozone-air mixture to a poultry house.
